(19) United States
(12) Patent Application Publication
(10) Pub. No.: US 2025/0109415 A1
(43) Pub. Date: Apr. 3, 2025

(54) SUSTAINABLE FUNCTIONALIZATION AND METHODS OF USE THEREOF

(71) Applicant: ARIZONA BOARD OF REGENTS ON BEHALF OF ARIZONA STATE UNIVERSITY, Scottsdale, AZ (US)

(72) Inventors: Logan Hessefort, Tempe, AZ (US); Kyle Biegasiewicz, Atlanta, GA (US)

(21) Appl. No.: 18/902,160

(22) Filed: Sep. 30, 2024

Related U.S. Application Data
(60) Provisional application No. 63/541,165, filed on Sep. 28, 2023.

(51) Int. Cl. C12P 7/20; C12N 9/18; C12N 9/64; C12P 13/02; C12P 19/02

(52) U.S. Cl. CPC C12P 7/20; C12N 9/18; C12N 9/6408; C12P 13/02; C12P 19/02; C12Y 301/01003; C12Y 304/21014

(57) ABSTRACT

The disclosure is drawn towards sustainable functionalization and methods of use thereof.

This application claims priority to U.S. Provisional Application No. 63/541,165 filed on Sep. 28, 2023, the entire contents of which are incorporated herein by reference.

### GOVERNMENT INTERESTS

This invention was made with government support under 2132183 awarded by the National Science Foundation. The government has certain rights in the invention.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE INVENTION

The disclosure is drawn towards sustainable functionalization and methods of use thereof.

### BACKGROUND OF THE INVENTION

Late-stage functionalization chemistry is utilized in the pharmaceutical industry, however, it is often challenging to discover highly selective late-stage functionalizations with little to no side product formation.

### SUMMARY OF THE INVENTION

Aspects of the invention are drawn towards a method of late-stage functionalization, the method comprising: selecting a compound with a nucleophilic moiety; reacting the nucleophilic moiety with a protecting group reagent, thereby producing an activated compound; subjecting the activated compound to at least one functionalization molecule and at least one hydrolase, wherein the functionalization molecule contains a nucleophilic group, thereby producing a transcarbamoylation reaction product. In embodiments, the method of late-stage functionalization comprises a reaction according to:

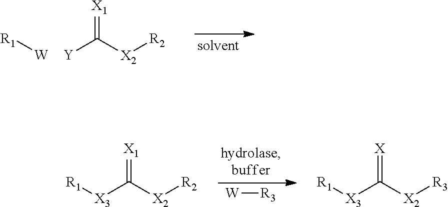

wherein R₁ is an organic moiety; R₂ is selected from the group consisting of -Ph, -Me, —CCH₂CCHCCH₂, -Et, —Pr, -Bu, or —CH₂Ph; R₃ is an organic moiety; W is selected from the group consisting of —NH₂, —SH, —OH or —NHR₄, wherein R₄ is selected from the group consisting of alkyl, alkenyl, alkynyl, aryl, heteroaryl, cycloalkyl, heterocycloalkyl, haloalkyl, hydroxyalkyl, alkoxyalkyl, carboxyalkyl, aminoalkyl, or a substituted derivative thereof; X₁ is selected from the group consisting of —O—, —S—, or —NH—; X₂ is selected from the group consisting of —O—, —S—, or —NH; X₃ is selected from the group consisting of —O—, —S—, —NH—, or —NR₄—, wherein R₄ is selected from the group consisting of alkyl, alkenyl, alkynyl, aryl, heteroaryl, cycloalkyl, heterocycloalkyl, haloalkyl, hydroxyalkyl, alkoxyalkyl, carboxyalkyl, aminoalkyl, or a substituted derivative thereof; wherein X₃ is —O— when W is —OH, wherein X₃ is —S— when W is —SH, and wherein X₃ is —NH when W is NH₂; and Y is selected from the group consisting of —Cl, —OPh, —OMe, —OCCH₂CCHCCH₂, —OEt, —OPr, —OBu, or —OCH₂Ph.

In embodiments, R₁ is selected from the group consisting of alkyl, alkenyl, alkynyl, aryl, heteroaryl, cycloalkyl, heterocycloalkyl, haloalkyl, hydroxyalkyl, alkoxyalkyl, carboxyalkyl, aminoalkyl, or a substituted derivative thereof. In embodiments, R₃ is selected from the group consisting of alkyl, alkenyl, alkynyl, aryl, heteroaryl, cycloalkyl, heterocycloalkyl, haloalkyl, hydroxyalkyl, alkoxyalkyl, carboxyalkyl, aminoalkyl, or a substituted derivative thereof. In embodiments, the hydrolase is selected from the group consisting of NovoCor® AD L, Alcalase® 2.4 L FG, Alcalase® 2.5 L, Esperase® 8.0 L, Savinase® 16 L, or a combination thereof. In embodiments, the nucleophilic moiety is selected from the group consisting of an amine, an alcohol, or a thiol. In embodiments, the protecting group reagent is selected from the group consisting of t-Butyloxycarbonyl chloride, di-tert-butyl decarbonate, benzyloxycarbonyl chloride, fluorenylmethyloxycarbonyl chloride, allyloxycarbonyl chloride, 2,2,2-Trichloroethoxycarbonyl chloride (Troc-Cl), methyl chloroformate, ethyl chloroformate, 1-Chloroethyl chloroformate, trichloromethyl chloroformate, chloromethyl chloroformate, dimethyl carbonate, (1S)-(+)-Menthyl chloroformate, phenyl chloroformate, or a combination thereof. In embodiments, the functionalization molecule is selected from the group consisting of an alcohol, a polyol, or a combination thereof. In embodiments, the alcohol is selected from the group consisting of methanol, ethanol, propanol, butanol, pentanol, hexanol, heptanol, octanol, nonanol, decanol, isoamyl alcohol, cyclohexanol, benzyl alcohol, allyl alcohol, propargyl alcohol, phenylethyl alcohol, methallyl alcohol, furfuryl alcohol, an isomer thereof, or a combination thereof. In embodiments, the polyol is selected from the group consisting of a diol, a triol, a sugar, a sugar alcohol, a polymeric polyol, or any combination thereof. In embodiments, the diol is selected from the group consisting of ethylene glycol, 1,2-propanediol, 1,3-propanediol, 1,3-butanediol, 1,4-butanediol. In embodiments, the triol is glycerol. In embodiments, the sugar alcohol is selected from the group consisting of sorbitol, mannitol, xylitol, or any combination thereof. In embodiments, the sugar is selected from the group consisting of glucose, fructose, maltose, or any combination thereof. In embodiments, the polymeric polyol is selected from the group consisting of polyethylene glycol, polyethylene glycol mono methyl ether, polypropylene glycol mono ethyl ether, polypropylene glycol, polypropylene glycol mono butyl ether, polypropylene glycol mono methyl ether, ethylene glycol mono-tert-butyl ether, polyethylene glycol monoethyl ether, or any combination thereof. In embodiments, the late-stage functionalized molecule is selected from the group consisting of a carbamate, a carbonate, a thiocarbonate, a urea, or a thiocarbamate. In embodiments, the method further comprises purifying the late-stage functionalized molecule. In embodiments, the method does not require a base catalyst.

Aspects of the disclosure are drawn towards a compound produced by a method described herein.

Aspects of the disclosure are drawn towards a pharmaceutical composition comprising a compound produced by a method described herein and a pharmaceutically acceptable excipient.

Other objects and advantages of this invention will become readily apparent from the ensuing description.

### BRIEF DESCRIPTION OF THE FIGURES

FIG. 1 shows non-limiting, exemplary schematic of the late-stage functionalization process described herein. The nucleophilic solvent comprises but is not limited to alcohols, amines, and thiols.

FIG. 2 shows non-limiting, exemplary substrates of the disclosure.

FIG. 3 shows non-limiting, exemplary late-stage functionalizations.

FIG. 4 shows a non-limiting, exemplary illustration of a bi-catalytic mechanism for hydrolysis by hydrolase enzymes.

FIG. 5 shows a non-limiting experimental design of a 3-dimensional surface plot indicating the relationship between temperature, ethylene glycol concentration, and yield of transcarbamoylated product at fixed 10 v/v % enzyme solution concentration. Further, the alcohol gold highlight saturation is based on their relative yields of transcarbamoylated product. Maroon highlights indicate side product formation.

FIG. 6 shows non-limiting exemplary ¹H-NMR spectra of enzymatic reactions in CDCl₃ at 500 MHz.

FIG. 7 shows non-limiting, stacked, exemplary high-pressure liquid chromatography traces from the same samples as FIG. 6.

FIG. 8 shows non-limiting, exemplary LCMS traces.

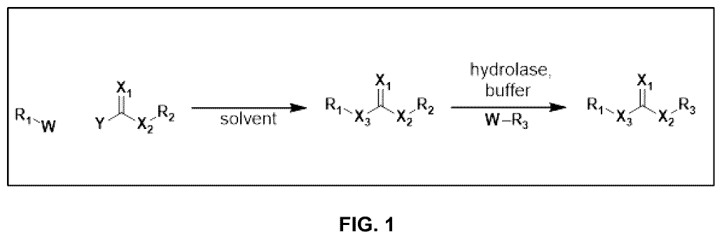

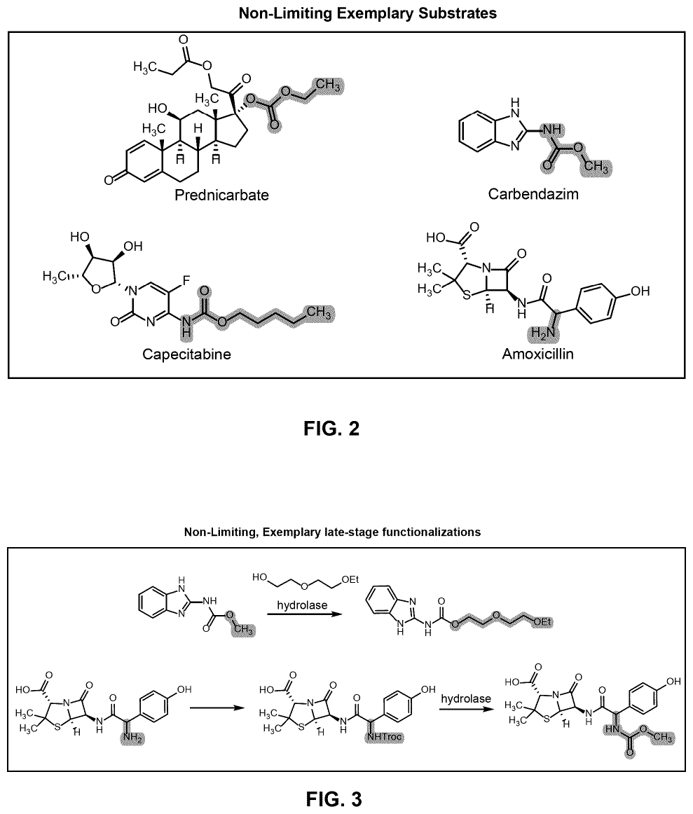

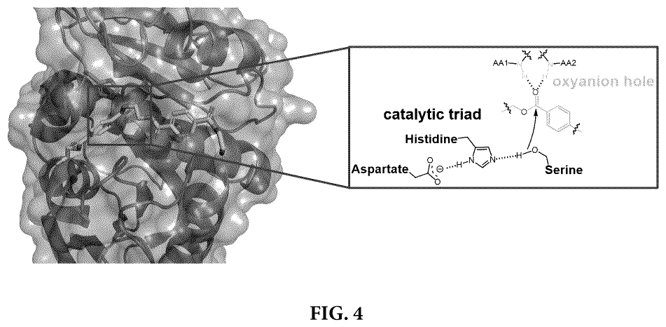

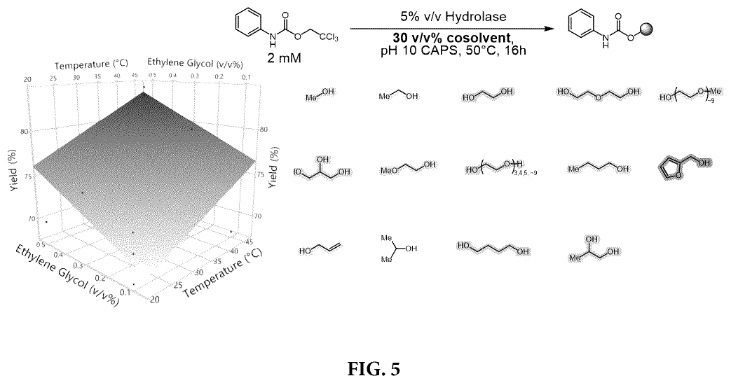

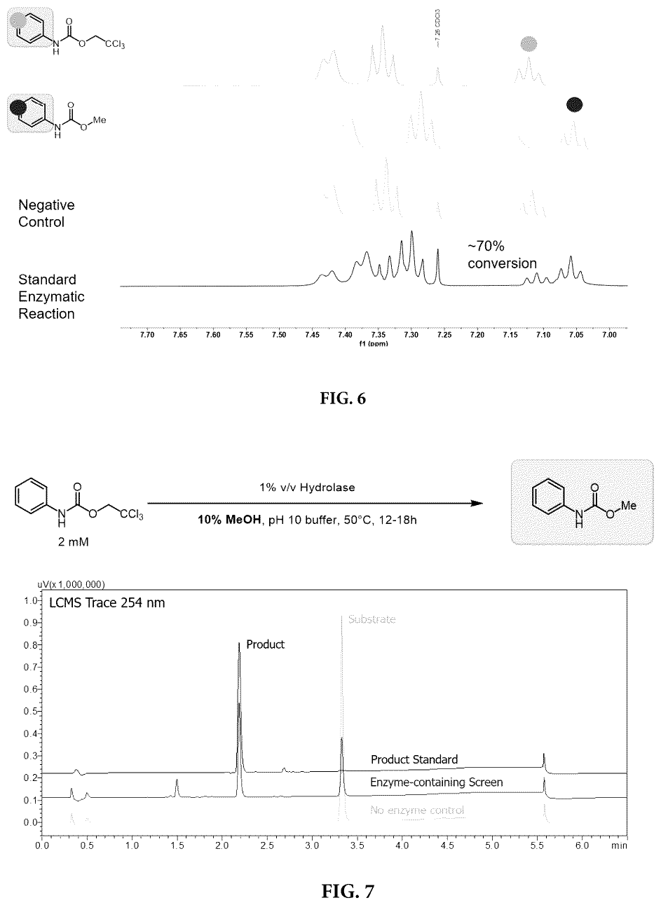

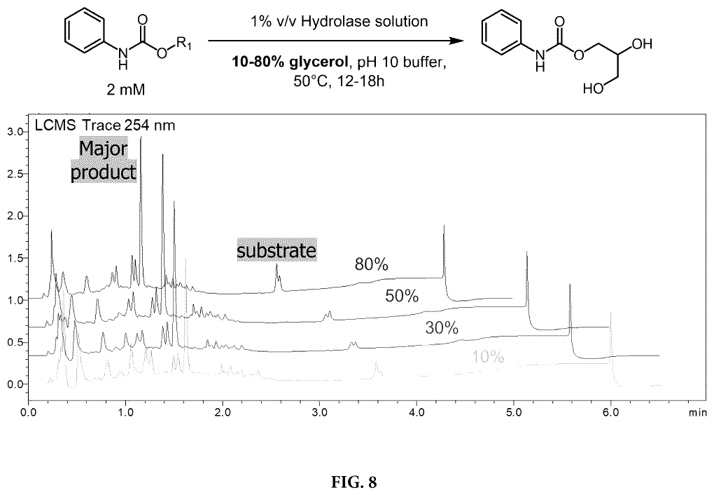

### DETAILED DESCRIPTION OF THE INVENTION

Detailed descriptions of one or more embodiments are provided herein. It is to be understood, however, that the invention can be embodied in various forms. Therefore, specific details disclosed herein are not to be interpreted as limiting, but rather as a basis for the claims and as a representative basis for teaching one skilled in the art to employ the invention in any appropriate manner.

The singular forms “a”, “an” and “the” include plural reference unless the context clearly dictates otherwise. The use of the word “a” or “an” when used in conjunction with the term “comprising” in the claims and/or the specification can mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein, the term “about” can refer to approximately, roughly, around, or in the region of When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower). In embodiments, the term “about” can be denoted by “˜”.

As used herein, the term “substantially the same” or “substantially” can refer to variability typical for a particular method is taken into account.

The terms “sufficient” and “effective”, as used interchangeably herein, can refer to an amount (e.g., mass, volume, dosage, concentration, and/or time period) needed to achieve one or more result(s).

Before explaining at least one embodiment of the disclosure in detail, it is to be understood that the disclosure is not necessarily limited in its application to the details set forth in the following description or exemplified by the examples. The disclosure can be used for other embodiments or of being practiced or carried out in various ways. Other compositions, compounds, methods, features, and advantages of the disclosure will be or become apparent to one having ordinary skill in the art upon examination of the following drawings, detailed description, and examples. All such additional compositions, compounds, methods, features, and advantages can be included within this description, and be within the scope of the disclosure.

Aspects of the invention are drawn towards a method of late-stage functionalization, comprising selecting a compound with a nucleophilic moiety; reacting the nucleophilic moiety with a protecting group reagent, thereby producing an activated compound; subjecting the activated compound to at least one functionalization molecule and at least one hydrolase enzyme, wherein the functionalization molecule contains a nucleophilic group, thereby producing a transcarbamoylation reaction product.

As used herein, the term “late-stage functionalization” can refer to the introduction of a functional group towards the end of a synthetic scheme and/or the introduction of a new functional group on a complex molecule. For example, the term “late-stage functionalization can refer to a chemoselective transformation performed on a complex molecule to provide at least one analog in sufficient quantity and purity for a given purpose, without requiring the addition of a functional group that solely serves to enable the transformation.

For example, late-stage functionalization can comprise PEGylation and bioconjugation.

As used herein, the term “functionalization molecule” can refer to the molecule that contains the functional group to be added to the initial compound with a nucleophilic moiety. For example, the term “functionalization molecule” can refer to a chemical compound that contains a functional group intended for attachment to another molecule (e.g., the substrate or target molecule) through a chemical reaction. This molecule can serve as the source of the new functional group that will be added to modify or enhance the properties of the target molecule.

As used herein, the term “protecting group reagent” can refer to a reagent which introduces a protecting group on a molecule. As used herein, the term “protecting group” can refer to a chemical functional group that is added to temporarily block or mask the reactivity of a functional group. For example, the protecting group can be any protecting group known in the art, such as a protecting group for amines, thiols, and alcohols. For example, the protecting group can be selected from the group consisting of t-Butyloxycarbonyl chloride, di-tert-butyl decarbonate, benzyloxycarbonyl chloride, fluorenylmethyloxycarbonyl chloride, allyloxycarbonyl chloride, 2,2,2-Trichloroethoxycarbonyl chloride (Troc-Cl), methyl chloroformate, ethyl chloroformate, 1-Chloroethyl chloroformate, trichloromethyl chloroformate, chloromethyl chloroformate, dimethyl carbonate, (1S)-(+)-Menthyl chloroformate, phenyl chloroformate, or a combination thereof.

As used herein, the term “activated compound” can refer to a molecule that has been modified to increase its reactivity. For example, the molecule can have increased susceptibility to nucleophilic attack. This activation can involve making the carbonyl group more electrophilic or creating a better-leaving group. Activated compounds can have enhanced electrophilicity at the carbonyl carbon, lower activation energy barriers, and increased reaction rates compared to their non-activated counterparts. The activation process aims to make the reaction proceed more efficiently under milder conditions.

In embodiments, the method of late-stage functionalization can comprise a reaction according to:

In embodiments, the method of late-stage functionalization comprises a reaction according to:

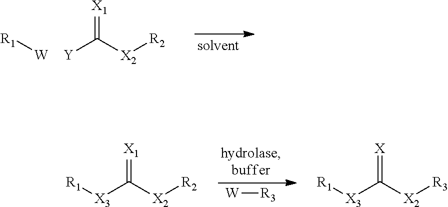

wherein R₁ is an organic moiety; R₂ is selected from the group consisting of -Ph, -Me, —CCH₂CCHCCH₂, -Et, —Pr, -Bu, or —CH₂Ph; R₃ is an organic moiety; W is selected from the group consisting of —NH₂, —SH, —OH or —NHR₄, wherein R₄ is selected from the group consisting of alkyl, alkenyl, alkynyl, aryl, heteroaryl, cycloalkyl, heterocycloalkyl, haloalkyl, hydroxyalkyl, alkoxyalkyl, carboxyalkyl, aminoalkyl, or a substituted derivative thereof; X₁ is selected from the group consisting of —O—, —S—, or —NH—; X₂ is selected from the group consisting of —O—, —S—, or —NH; X₃ is selected from the group consisting of —O—, —S—, —NH—, or —NR₄—; wherein X₃ is —O— when W is —OH, wherein X₃ is —S— when W is —SH, and wherein X₃ is —NH when W is NH₂; and Y is selected from the group consisting of —Cl, —OPh, —OMe, —OCCH₂CCHCCH₂, —OEt, —OPr, —OBu, or —OCH₂Ph.

In embodiments, R₁ is selected from the group consisting of alkyl, alkenyl, alkynyl, aryl, heteroaryl, cycloalkyl, heterocycloalkyl, haloalkyl, hydroxyalkyl, alkoxyalkyl, carboxyalkyl, aminoalkyl, or a substituted derivative thereof.

In embodiments, R₃ is selected from the group consisting of alkyl, alkenyl, alkynyl, aryl, heteroaryl, cycloalkyl, heterocycloalkyl, haloalkyl, hydroxyalkyl, alkoxyalkyl, carboxyalkyl, aminoalkyl, or a substituted derivative thereof.

In embodiments, the R₁ can be an aromatic group. As used herein, the term “aromatic” can refer to moieties that satisfy the Huckel 4n+2 rule for aromaticity and comprises both aryl and heteroaryl compounds. For example, the methods described herein can comprise a reaction according to:

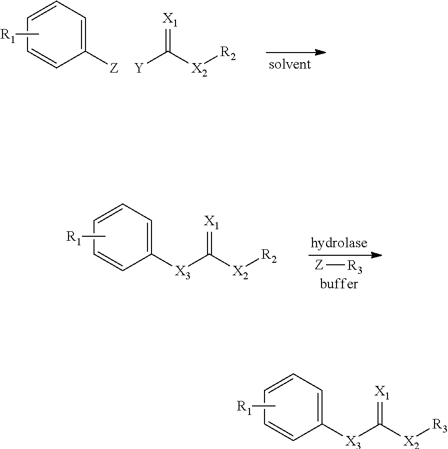

wherein R₁ is an organic moiety; R₂ is selected from the group consisting of -Ph, -Me, —CCH₂CCHCCH₂, -Et, —Pr, -Bu, or —CH₂Ph; R₃ is an organic moiety; Z is selected from the group consisting of —NH₂, —SH, or —OH; X₁ is selected from the group consisting of —O—, —S—, or —NH—; X₂ is selected from the group consisting of —O—, —S—, or —NH; X₃ is selected from the group consisting of —O—, —S—, or —NH, wherein X₃ is —O— when Z is —OH, wherein X₃ is —S— when Z is —SH, and wherein X₃ is —NH when Z is NH₂; and Y is selected from the group consisting of —Cl, —OPh, —OMe, —OCCH₂CCHCCH₂, —OEt, —OPr, —OBu, or —OCH₂Ph.

In embodiments, the organic moiety can be an aliphatic group. In embodiments, the aliphatic group can be saturated or unsaturated. In embodiments, the aliphatic group can be branched or unbranched.

As used herein, the term “functional group” can refer to any functional group known in the art. For example, the functional groups can be determined by one of ordinary skill in the art using any method known in the art. As used herein, the term “moiety” can refer to a functional group, a segment of a molecule, or a combination of functional groups. In embodiments, the terms “functional group” and “moiety” can be used interchangeably. For example, the functional groups or moieties described herein can be one or more alkyl groups. The term “alkyl” can refer to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. For example, in carbamate chemistry, alkyl groups typically contain about 1 to about carbon atoms in their backbone.

In some embodiments, a straight chain or branched chain alkyl can have 20 or fewer carbon atoms in its backbone (e.g., C₁-C₂₀ for straight chains, C₃-C₂₀ for branched chains), 20 or fewer, 12 or fewer, or 7 or fewer. Likewise, in some embodiments cycloalkyls can have from 3-10 carbon atoms in their ring structure, e.g., have 5, 6 or 7 carbons in the ring structure. The term “alkyl” (or “lower alkyl”) as used throughout the specification, examples, and claims can include both “unsubstituted alkyls” and “substituted alkyls”, the latter of which refers to alkyl moieties having one or more substituents replacing a hydrogen on one or more carbons of the hydrocarbon backbone. Such substituents include, but are not limited to, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, a hosphinate, amino, amido, amidine, imine, cyano, nitro, azido, sulfhydryl, alkylthio, sulfate, sulfonate, sulfamoyl, sulfonamido, sulfonyl, heterocyclyl, aralkyl, or an aromatic or heteroaromatic moiety. Without wishing to be bound by theory, the substituents can be any substituent that does not significantly interfere with the intended function or reactivity of the carbonyl compound.

Unless the number of carbons is otherwise specified, “lower alkyl” as used herein can refer to an alkyl group, as defined herein, but having from one to ten carbons, or from one to six carbon atoms in its backbone structure. Likewise, “lower alkenyl” and “lower alkynyl” have similar chain lengths. In some embodiments, alkyl groups are lower alkyls. In some embodiments, a substituent described herein as alkyl can be a lower alkyl.

It will be understood by those skilled in the art that the moieties substituted on the hydrocarbon chain can themselves be substituted, if appropriate. For instance, the substituents of a substituted alkyl can include halogen, hydroxy, nitro, thiols, amino, azido, imino, amido, phosphoryl (including phosphonate andphosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Cycloalkyls can be substituted in the same manner.

In embodiments, the functional groups or moieties described herein can refer to one or more heteroalkyl groups. The term “heteroalkyl”, as used herein, refers to straight or branched chain, or cyclic carbon-containing radicals, or combinations thereof, containing at least one heteroatom. Suitable heteroatoms include, but are not limited to, O, N, Si, P, Se, B, and S, wherein the phosphorous and sulfur atoms are optionally oxidized, and the nitrogen heteroatom is optionally quaternized. Heteroalkyls can be substituted as defined herein for alkyl groups.

In embodiments, the functional groups or moieties described herein can refer to one or more alkylthio groups. The term “alkylthio” refers to an alkyl group, as defined herein, having a sulfur radical attached thereto. In some embodiments, the “alkylthio” moiety is represented by one of —S-alkyl, —S-alkenyl, and —S-alkynyl. Representative alkylthio groups include methylthio, and ethylthio. The term “alkylthio” also encompasses cycloalkyl groups, alkene and cycloalkene groups, and alkyne groups. “Arylthio” refers to aryl or heteroaryl groups. Alkylthio groups can be substituted as defined herein for alkyl groups.

In embodiments, the functional groups or moieties described herein can refer to one or more alkenyl and alkynyl groups. The terms “alkenyl” and “alkynyl”, refer to unsaturated aliphatic groups analogous in length and possible substitution to the alkyls described herein, but that contain at least one double or triple bond respectively. In embodiments, the alkenyl and alkynyl groups can be limited to those that do not interfere with carbonyl reactivity.

In embodiments, the functional groups or moieties described herein can refer to one or more alkoxyl groups. The terms “alkoxyl” or “alkoxy” as used herein refers to an alkyl group, as defined herein, having an oxygen radical attached thereto. Representative alkoxyl groups include methoxy, ethoxy, propyloxy, and tert-butoxy. An “ether,” for example, can be two hydrocarbons covalently linked by an oxygen. Accordingly, the substituent of an alkyl that renders that alkyl an ether is or resembles an alkoxyl, such as can be represented by one of —O— alkyl, —O-alkenyl, and —O-alkynyl. Aroxy can be represented by —O-aryl or O-heteroaryl, wherein aryl and heteroaryl are as defined herein. The alkoxy and aroxy groups can be substituted as described herein for alkyl.

In embodiments, the functional group or moiety described herein can be an alkyl group, an amine, an imide, a halogen, an aryl, a sulfhydryl, a hydroxyl, a sulfonyl group, or any combination of groups described herein. As used here, the terms “amine” and “amino” are art-recognized and can refer to both unsubstituted and substituted amines, e.g., a moiety that can be represented by the general formula:

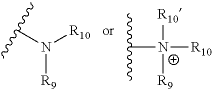

wherein R₉, R₁₀, and R_{10′} each independently represent a hydrogen, an alkyl, an alkenyl, —(CH₂)ₘ—Rs or R₉ and R₁₀ taken together with the N atom to which they are attached complete a heterocycle having from 4 to 8 atoms in the ring structure; Rs represents an aryl, a cycloalkyl, a cycloalkenyl, a heterocycle or a polycycle; and m is zero or an integer in the range of 1 to 8. In some embodiments, only one of R₉ or R₁₀ can be a carbonyl, e.g., R₉, R₁₀ and the nitrogen together do not form an imide. In still other embodiments, the term “amine” does not encompass amides, e.g., wherein one of R₉ and R₁₀ represents a carbonyl. In additional embodiments, R₉ and R₁₀ (and optionally R₁₀,) each independently represent a hydrogen, an alkyl or cycloalkyl, an alkenyl or cycloalkenyl, or alkynyl. Thus, the term “alkylamine” as used herein can refer to an amine group, having a substituted (as described herein for alkyl) or unsubstituted alkyl attached thereto, i.e., at least one of R₉ and R₁₀ is an alkyl group. In embodiments, the functional group or moiety can comprise an aryl group.

As used herein, “aryl” can refer to a monovalent aromatic hydrocarbon radical derived by the removal of one hydrogen atom from a single carbon atom of a parent aromatic ring system. Non-limiting, exemplary aryl groups comprise groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like as known in the art.

As used herein, the term “imide” can refer to —C(O)NR′R″, wherein R′ and R″ are each independently hydrogen, or a substituted or unsubstituted alkyl, cycloalkyl, alkenyl, alkynyl, aryl aralkyl, heterocyclyl or heterocyclylalkyl group as defined herein.

As used herein, the term “halogen” can refer to —F, —Cl, —Br or —I; the term “sulfhydryl” can refer to —SH; the term “hydroxyl” can refer to —OH; and the term “sulfonyl” can refer to —SO₂—.

In embodiments, the functional group or moiety described herein can be a substituted functional group or moiety. The term “substituted” as used herein, refers to substituents of the compounds described herein. In the broadest sense, the permissible substituents comprise acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms, for example 1-14 carbon atoms, and can comprise one or more heteroatoms such as oxygen, sulfur, or nitrogen grouping in linear, branched, or cyclic structural formats.

Representative substituents comprise alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, amino acid, peptide, and polypeptide groups. As used herein in reference to an “R” group, the name used to describe said “R” group can be the chemical name prior to the removal of a hydrogen. For example, wherein “R” is described as an “alkane” can refer to an “alkyl” group.

Heteroatoms such as nitrogen can have hydrogen substituents and/or substituents of organic compounds described herein which satisfy the valences of the heteroatoms. It is understood that “substitution” or “substituted” comprises substitutions is accordance with permitted valence of the substituted atom and the substituent, and that the substitution results in a compound that does not spontaneously undergo transformation such as by rearrangement, cyclization, elimination, etc.

In a broad aspect, the substituents can comprise acyclic and cyclic, branched and unbranched, carbocyclic and heterocyclic, aromatic and nonaromatic substituents of organic compounds. Illustrative substituents include, for example, those described herein. The permissible substituents can be one or more and the same or different for appropriate organic compounds. The heteroatoms for example, nitrogen can have hydrogen substituents and/or substituents of organic compounds described herein which satisfy the valencies of the heteroatoms.

Without wishing to be bound by theory, the substituent can be selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, and thioketone, each of which optionally is substituted with one or more suitable substituents. In some embodiments, the substituent is selected from alkoxy, aryloxy, alkyl, alkenyl, alkynyl, amide, amino, aryl, arylalkyl, carbamate, carboxy, cycloalkyl, ester, ether, formyl, haloalkyl, heteroaryl, heterocyclyl, ketone, phosphate, sulfide, sulfinyl, sulfonyl, sulfonic acid, sulfonamide, and thioketone, wherein each of the alkoxy, aryloxy, alkyl, alkenyl, alkynyl, amide, amino, aryl, arylalkyl, carbamate, carboxy, cycloalkyl, ester, ether, formyl, haloalkyl, heteroaryl, heterocyclyl, ketone, phosphate, sulfide, sulfinyl, sulfonyl, sulfonic acid, sulfonamide, and thioketone can be further substituted with one or more substituents. However, one of ordinary skill in the art, can limit substituents based upon their effect on the carbonyl compound's function or reactivity.

Examples of substituents include, but are not limited to, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, thioketone, ester, heterocyclyl, —CN, aryl, aryloxy, perhaloalkoxy, aralkoxy, heteroaryl, heteroaryloxy, heteroarylalkyl, heteroaralkoxy, azido, alkylthio, oxo, acylalkyl, carboxy esters, carboxamido, acyloxy, aminoalkyl, alkylaminoaryl, alkylaryl, alkylaminoalkyl, alkoxyaryl, arylamino, aralkylamino, alkylsulfonyl, carboxamidoalkylaryl, carboxamidoaryl, hydroxyalkyl, haloalkyl, alkylaminoalkylcarboxy, aminocarboxamidoalkyl, cyano, alkoxyalkyl, perhaloalkyl, arylalkyloxyalkyl, and the like. In some embodiments, the substituent is selected from cyano, halogen, hydroxyl, and nitro.

In embodiments, the enzyme comprises a hydrolase or any enzyme capable of hydrolyzing a carbonyl containing compound. For example, the hydrolase can comprise proteases (subtilisins) and estersases. For example, the hydrolase can comprise NovoCor® AD L, Alcalase® 2.4 L FG, Alcalase® 2.5 L, Esperase® 8.0 L, or Savinase® 16 L.

In embodiments, the nucleophilic moiety is selected from the group consisting of an amine, an alcohol, or a thiol. In embodiments, the functionalization molecule is selected from the group consisting of an alcohol, a polyol, or a combination thereof. For example, the alcohol can be selected from any alcohol know in the art. For example, the alcohol can be methanol, ethanol, propanol, butanol, pentanol, hexanol, heptanol, octanol, nonanol, decanol, isoamyl alcohol, cyclohexanol, benzyl alcohol, allyl alcohol, propargyl alcohol, phenylethyl alcohol, methallyl alcohol, furfuryl alcohol, an isomer thereof, or a combination thereof.

As used herein, the term “polyol” can refer to any compound that contains more than one alcohol functional group. For example, the polyol can include, but is not limited to, a diol, a triol, a sugar, a sugar alcohol, a polymeric polyol, or any combination thereof. In embodiments, the diol can be any diol known in the art. For example, the diol can be ethylene glycol, 1,2-propanediol, 1,3-propanediol, 1,3-butanediol, 1,4-butanediol. In embodiments, the triol can be any triol known in the art. For example, the triol is glycerol. In embodiments, the sugar alcohol can be any sugar alcohol known in the art. For example, the sugar alcohol can include, but is not limited to, sorbitol, mannitol, xylitol, or any combination thereof. In embodiments, the sugar can be any sugar known in the art. For example, the sugar can include, but is not limited to, glucose, fructose, maltose, or any combination thereof. In embodiments, the polymeric polyol can be any polymeric polyol known in the art. For example, the polymer polyol can include, but is not limited to, polyethylene glycol, polyethylene glycol mono methyl ether, polypropylene glycol mono ethyl ether, polypropylene glycol, polypropylene glycol mono butyl ether, polypropylene glycol mono methyl ether, ethylene glycol mono-tert-butyl ether, polyethylene glycol monoethyl ether, or any combination thereof.

In embodiments, the late-stage functionalized molecule is selected from the group consisting of a carbamate, carbonate, thiocarbonate, or thiocarbamate. Non-limiting, exemplary advantages of the methods described herein are high activated compound conversion, mild conditions, and the wide range of library of nucleophilic reagents that can be used in late-stage functionalization, including but not limited to alcohols, thiols, and amines. For example, other approaches are limited to the finite number of available carbonates (see, e.g., Meinert et al., Angew. Chem. Int. Ed. 2024, e202405152).

In embodiments, the method can further comprise purifying the late-stage functionalized molecule. For example, the purification can comprise any purification process know in the art. For example, the purification process can comprise crystallization, chromatography, filtration, liquid-liquid extraction, precipitation, and distillation.

In embodiments, the method does not require a base catalyst. For example, the reaction can be catalyzed solely by a biocatalyst.

As used herein, the term “transcarbamoylation” can refer to the transfer of a carbamoyl group to another molecule. As used herein, the term “carbmoyl” can refer to a functional group comprising (NH₂—CO—).

Aspects of the invention are drawn towards a compound produced by any one of the methods described herein.

Aspects of the invention are drawn towards a pharmaceutical composition comprising the compound produced by any one of the methods described herein and a pharmaceutically acceptable excipient. A “pharmaceutically acceptable excipient,” “pharmaceutically acceptable diluent,” “pharmaceutically acceptable carrier,” or “pharmaceutically acceptable adjuvant” can refer to an excipient, diluent, carrier, and/or adjuvant that are useful in preparing a pharmaceutical composition that are generally safe, non-toxic and neither biologically nor otherwise undesirable, and include an excipient, diluent, carrier, and adjuvant that are acceptable for veterinary use and/or human pharmaceutical use. “A pharmaceutically acceptable excipient, diluent, carrier and/or adjuvant” as used herein can include one and more such excipients, diluents, carriers, and adjuvants.

The phrase “pharmaceutical composition” or a “pharmaceutical formulation” can refer to a composition or pharmaceutical composition suitable for administration to a subject, such as a mammal, especially a human and that can refer to the combination of an active agent(s), or ingredient with a pharmaceutically acceptable carrier or excipient, making the composition suitable for diagnostic, therapeutic, or preventive use in vitro, in vivo, or ex vivo. A “pharmaceutical composition” can be sterile and can be free of contaminants that can elicit an undesirable response within the subject (e.g., the compound(s) in the pharmaceutical composition is pharmaceutical grade). Pharmaceutical compositions can be designed for administration to subjects or patients in need thereof via a number of different routes of administration including oral, intranasal, topical, intravenous, buccal, rectal, parenteral, intraperitoneal, intradermal, intracheal, intramuscular, subcutaneous, by stent-eluting devices, catheters-eluting devices, intravascular balloons, inhalational and the like.

### EXAMPLES

Examples are provided herein to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

### Biocatalytic Transcarbamoylation for Late-Stage Functionalization

This disclosure describes a new, integrated, and enzymatic approach to a highly efficient late-stage functionalization method through catalytic transcarbamoylation for the modification of compounds in cosmetics, pharmaceuticals, and agrochemicals, widening its market applicability. The biocatalytic process underscores a commitment to sustainability and circularity within the polyurethane sector, with direct implications for significant cost savings, increased scalability, and robust market potential. This disclosure has applications in environmental stewardship, cost-efficient operations, and functionalization solutions.

### BACKGROUND

The disclosure is drawn towards the fields of late-stage functionalization, for example, on late-stage enhancement of pharmaceuticals. Late-stage functionalization chemistry is utilized in the pharmaceutical industry, however, it is often challenging to discover highly selective late-stage functionalizations with little to no side product formation.

In the realm of late-stage functionalization, PEGylation—the attachment of polyethylene glycol (PEG) or methoxy PEG (mPEG) to improve the solubility, stability, and therapeutic efficacy of compounds in pharmaceuticals and cosmetics—has been of interest. Yet, conventional PEGylation methods have limitations, including the susceptibility of the ester and amide linkages to hydrolysis in vivo. Multiple widely prescribed pharmaceuticals, such as Mircera and Ozempic contain ethylene glycol units, methoxy-PEG and diethylene glycol, respectively, and both are attached to the drugs with an amide functionality. Carbamates are more hydrolytically stable than esters and amides in vivo because few enzymes can catalyze their hydrolysis, whereas hydrolases can catalyze the hydrolysis of esters and amides. New methodology described herein adds carbamate PEGylation and general carbamate addition to pharmaceuticals in a simple and efficient manner.

Traditional carbamate synthesis is performed using hazardous isocyanates, bromine reagents, or explosive azides. The method described herein allows for the selective catalytic formation of carbamates in a late-stage functionalization which can be used in the synthesis of carbonyl-containing drugs, cosmetic ingredients, and agrochemicals. For example, many insecticides, such as carbaryl and Propoxur, contain carbamate functionalities. Further, this disclosure provides a method of rapid and simple generation of new active pharmaceutical ingredients (APIs) and agrochemicals with enhanced properties over their amide or ester-containing counterparts.

### Non-Limiting Summary:

This disclosure describes, without wishing to be bound by theory, an enzymatic method for a selective late-stage functionalization process through catalytic transcarbamoylation, offering an efficient and sustainable solution for the challenges of catalytic modification of compounds. Non-limiting examples of compounds comprise compounds in the cosmetic, pharmaceuticals, and agrochemicals industries. For example, the method described herein can employ hydrolase enzymes, for example commercially available endoproteases. Further, we have performed catalytic transcarbamoylation on small molecules. Notably, our approach operates under milder conditions than traditional transcarbamoylation techniques, reducing the need for high temperatures and toxic catalysts.

Our late-stage carbamate functionalization process can comprise a target molecule containing a nucleophilic functional group, for example an amine, but this process can be expanded functional groups such as alcohols, and thiols, which is reacted with 2,2,2-trichloroethoxycarbonyl chloride (Troc-Cl) or other carbamate-generating agents to form an activated carbamate. The activated alcohol detached from the carbamate and the carbamate is then transferred to another molecule such as methoxypolyethylene glycol (mPEG) molecule by the enzyme. Although PEGylation is one of the more readily relevant to the pharmaceutical industry, in theory, our method can transfer any alcohol or polyol onto the carbamate. This can allow for significant and stable late-stage diversification of molecular targets. Further, this method has the capability to be expanded to using amines as carbamate acceptors to generate ureas.

The non-limiting, exemplary process of Enzymatic PEGylation involves several steps:
1. Target Molecule Selection and Preparation: The process begins with a molecule featuring a nucleophilic functional group. While amines can be used to generate carbamates, other nucleophilic functional groups, such as alcohols and thiols can be accommodated to generate carbonates, thiocarbonates, or thiocarbamates.
2. Activation: The selected nucleophilic group is reacted with a carbamate-generating agent, such as 2,2,2-trichloroethoxycarbonyl chloride (Troc-Cl), to form an activated carbamate. The compounds are then purified, partially purified or left unpurified before the next step.
3. Enzymatic Transcarbamoylation: Tailored hydrolase enzymes (or a cocktail of multiple enzymes) are introduced, facilitating the transfer of the activated alcohol moiety attached to the carbamate to a polyethylene glycol (PEG) molecule or another nucleophile. Optimization of enzyme loading is an essential aspect of this step. The transcarbamoylation reaction is conducted under optimized conditions. The specific conditions such temperature, reactant ratio, and water content will be determined based on the unique properties of the system.
4. Product Separation: After the reaction, the product is separated and purified through any number of Purification procedures.

### Non-Limiting Summary:

This disclosure describes a biocatalytic platform for late-stage chemical functionalization. This platform uses an efficient and sustainable process to facilitate late-stage diversification through transcarbamoylation. Operating under mild conditions, this process negates the need for high temperatures and toxic catalysts, positioning it as a superior, environmentally conscious, and safer alternative. It indicates a significant leap towards eco-friendly practices and operator safety in the polymer industry. The applications of the methods described herein can permeate various sectors, including pharmaceuticals, cosmetics, and agrochemicals. This transformative technology employs commercially available biocatalysts to drive value across industries by, for example, enhancing product performance and synthesizing new compounds.

This disclosure can deliver significant commercial value by offering a comprehensive, cost-effective, and sustainable solution for managing performing selective late-stage functionalization. It champions economic and environmental benefits by enabling industries to streamline operations, minimize waste, and maximize resource utilization.

### Non-Limiting New Components of the Invention:

Enzymatic Approach: An innovative aspect of this disclosure is the utilization of readily available hydrolase enzymes for transcarbamoylation. This contrasts sharply with industry norms of employing high temperatures and harmful catalysts such as alkoxides and organotins.

Milder Operational Conditions: In contrast to traditional transcarbamoylation processes necessitating high temperatures (exceeding 100° C. often) and strong alkalinity (pH>14), our method operates under markedly milder conditions—around 50° C. and pH 10. These milder conditions increase the methods functional group tolerance.

Versatility in Late-stage Functionalization: Our innovation provides a broad array of late-stage functionalization reactions, for example PEGylation. PEGylation is a widely employed technique in medicinal chemistry, used to enhance the stability and efficacy of active pharmaceutical ingredients (APIs). Such functionality is not intrinsic to existing technologies, which broadens the application scope of our invention beyond polyurethane recycling, extending to various fields such as pharmaceuticals, agrochemistry, and cosmetics.

### Non-Limiting Advantages Over Current Technology and Impact:

Enzymatic Transcarbamoylation: Unlike conventional methods that often employ toxic or hazardous catalysts, our disclosure capitalizes on the catalytic activity of hydrolase enzymes. This biological approach provides a safer, more sustainable, and biocompatible alternative, minimizing health and environmental risks.

Operational Efficiency: Our method operates under milder conditions than traditional processes. Conventional transcarbamoylation often requires extreme conditions, such as temperatures exceeding 100° C. and high alkalinity (pH>14). In contrast, our method functions efficiently at approximately 50° C. and pH 10, significantly reducing energy demands, enhancing functional group tolerance, and lowering overall operational costs.

Versatility and Broad Applicability: This disclosure describes diverse late-stage functionalization reactions, including the pharmaceutical technique of PEGylation. This versatile capability allows for enhanced late-stage functionalization and diversification, benefiting sectors such as pharmaceuticals, agrochemistry, and cosmetics.

### Non-Limiting Commercial Value:

Pharmaceuticals: Our method provides a new approach for late-stage functionalization and diversification, for example PEGylation, opening opportunities for pharmaceutical manufacturers to optimize the solubility, stability, and potency of their drug formulations.

Cosmetics: Our invention can have applications in the cosmetic industry. By enhancing product properties through optimized PEGylation, cosmetic manufacturers can reap benefits. With constant demand for innovative and effective products in this industry, our method offers a valuable edge.

Agrochemistry: Our technology can be employed in the agrochemical industry to modify active ingredients for better performance. The improved delivery and effectiveness of agrochemical products through PEGylation can be attractive for this sector.

The competitive landscape for this technology can include conventional chemical processes used for late-stage functionalization. However, these traditional methods often demand harsh conditions and utilize hazardous chemicals, while our invention offers a milder, eco-friendlier alternative. The capability to perform diverse types of late-stage functionalization further distinguishes our method from existing technologies.

Moreover, the commercial potential of our invention is not confined to these industries. Its adaptability and sustainability can be valuable across various sectors seeking to innovate their products or processes with a conscious eye towards the environment.

Non-limiting Surprising Features: This disclosure describes a new enzymatic method for late-stage functionalization processes via transcarbamoylation and related similar reactions (e.g., other transesterification-type reactions) as known in the art. Unlike the existing methods that rely heavily on high temperatures and hazardous catalysts, this invention exploits the properties of hydrolase enzymes, for example endoproteases, for the transcarbamoylation reaction. Additionally, our method is distinctive in its operational parameters, capable of functioning under considerably milder conditions, thereby significantly reducing energy demands and environmental impact.

The process described herein indicates utility in several domains. For example, it describes a new method for performing a variety of late-stage functionalization reactions, for example PEGylation, thereby extending its applicability to pharmaceutical, agrochemical, and cosmetics sectors.

Hydrolase enzymes to facilitate the transcarbamoylation reaction is a surprising finding. For example, carbamates are extremely rare in nature, and transcarbamoylation was an undiscovered activity of the enzymes. We screened 10s of enzymes, under hundreds of conditions, with 10s of substrates to discover this activity. This disclosure redefines the applications of these enzymes, extending their known activities to the realm diverse chemical synthesis—a leap not indicated by their traditional classification. The ability of these enzymes to operate under milder conditions compared to traditional techniques while maintaining efficacy and selectivity is also unexpected. Furthermore, the enhancement of the properties of pharmaceuticals and cosmetics can be beyond the anticipation of existing technologies, thus solidifying the non-obvious nature of this invention.

Our disclosure comprises broad ranges of operational conditions and reaction parameters which can be modified for a variety of applications by one of ordinary skill in the art.

### Non-Limiting Examples

### Synthesis of the Troc-Protected Medicinally Relevant Compound

A 50 mL round-bottom flask is flame-dried, and 10 g (0.09 mol) of 4-amino phenol is added, along with 1.47 grams (5 mol %) of tetrabutylammonium bromide. The two solids are mixed thoroughly, and the reaction vessel is flushed with dry nitrogen gas to create an inert atmosphere. Subsequently, 20.4 g or 13.3 mL (1.05 equivalents) of 2,2,2-trichloroethyl chloroformate (Troc-Cl) is added dropwise at a rate of approximately 2 mL per minute to the solid mixture, with continued magnetic stirring. The reaction mixture is allowed to stir for an additional 30 minutes after the dropwise addition is complete. Afterward, a dilute solution of acetic acid (5 v/v %) is added to quench the reaction, and the product is extracted into ethyl acetate (3×30 mL). The combined organic layer is washed with brine, dried over anhydrous sodium sulfate, and concentrated under reduced pressure to obtain the product. Purity is determined by reverse-phase analytical liquid chromatography-mass spectrometry using a Shimadzu LCMS2020 with a phenyl stationary phase and an acetonitrile:water mobile phase. The product can be found to be >95% pure without the need for further purification.

### Biocatalytic Late-Stage Functionalization

142 mg (0.5 mmol) of the Troc-protected 4-amino phenol is separately dissolved into 3 mL of each of four different alcohols (methoxy polyethylene glycol 400, propylene glycol, ethylene glycol, and diethylene glycol) at 50° C. with shaking until all solid has fully dissolved. In a 20 mL glass vial equipped with a magnetic stir bar, a 7 mL solution is prepared, comprising 10 v/v % Savinase® 16 L, 25 mM pH 10 CAPS/NaOH buffer, and 2.5 mM (5 mol %) tetrabutylammonium bromide. The 3 mL of substrate dissolved in one of the respective alcohols is then added to make up a total of 10 mL, and the reaction is allowed to run at 50° C. with stirring until the conversion has reached >90% (for example, 24-72 h), as determined by reverse-phase liquid chromatography-mass spectrometry on a Shimadzu LCMS2020. Subsequently, dilute (5 v/v %) acetic acid solution is added to the reaction mixture, and the product is extracted into ethyl acetate (3×30 mL). The combined organic layer is washed with brine, dried over anhydrous sodium sulfate, and concentrated under reduced pressure. Purity can be determined by reverse-phase analytical liquid chromatography-mass spectrometry using a Shimadzu LCMS2020 with a phenyl stationary phase and an acetonitrile:water mobile phase. The product can be found to be about >90% pure without further purification. This method can be repeated with each alcohol, affording four carbamate analogs to acetaminophen.

Further, reaction conditions such as buffer composition, pH, and temperature can be optimized using methods described in the art by one of ordinary skill in the art. In silico tools such as design of experiments, density functional theory, and in vitro kinetics experiments can aide in guiding the temperature and pH at which the enzymes can be engineered to function at by determining activation energy barriers and equilibrium constants.

### Biocatalytic Late-Stage Functionalization:

Compounds containing carbamates can have biological activity, with several carbamate-containing natural products having been identified.^{10,11} As bioisosteres for peptide bonds, carbamates confer protease resistance and enhanced stability against enzymatic hydrolysis. Their lipophilicity also boosts drug membrane permeability. The capacity for hydrogen bonding and their conformational rigidity due to partial double bond character of the CO—N bond render them optimal for molecular recognition and enhanced binding affinity. Late-stage functionalization of medicinal compounds can allow for rapid and diverse modification of complex molecules, enabling efficient optimization of their pharmacological properties without synthesizing them from scratch.

Late-stage functionalization of medicinal compounds facilitates the swift and varied modification of intricate molecules, streamlining the optimization of their pharmacological properties without the need for ground-up synthesis. Our biocatalytic transcarbamoylation technique can provide a way to shield amines using a conventional carbamate protecting group and provides a gentle approach for swapping the carbamate ester with a variety of alcohols. This modular approach facilitates the diversification of an amine into various carbamates, which can indicate improved biological activity.

For example, described herein, we can optimize conditions such as temperature, buffer, pH, and enzyme concentration for transcarbamoylation activity with any enzyme described herein as determined by one of ordinary skill in the art by any method known in the art. For example, Savinase 16L through the use of Design of Experiments methods. These conditions can be tuned based upon increased or decreased alcohol concentrations as determined by one of ordinary skill in the art. Without wishing to be bound by theory, this can arise from enzyme denaturation or other factors. As a result, to achieve optimal yields with these wild-type enzymes, reaction conditions can be tailored for each specific enzyme, substrate, and alcohol using methods known in the art by one of ordinary skill in the art. The efficiency of TBAB can be gauged by the substrate's solubility in the chosen alcohol; when solubility is low, a PTC can become indispensable.

### Biocatalytic Late-Stage Functionalization:

Reactions conditions described herein can be determined and modified by one of ordinary skill in the art by any method known in the art. Therefore, reaction conditions (e.g., molar ratios of reagents, temperatures, reaction times) not explicitly enumerated herein are embodiments of the disclosure that can be determined by one of ordinary skill in the art.

Non-limiting, suitable amine protecting group agents can comprise t-Butyloxycarbonyl chloride, di-tert-butyl decarbonate, benzyloxycarbonyl chloride, fluorenylmethyloxycarbonyl chloride, allyloxycarbonyl chloride, 2,2,2-Trichloroethoxycarbonyl chloride, methyl chloroformate, ethyl chloroformate, 1-Chloroethyl chloroformate, trichloromethyl chloroformate, chloromethyl chloroformate, (1S)-(+)-Menthyl chloroformate, and phenyl chloroformate. In embodiments the protecting groups comprise 2,2,2-Trichloroethoxycarbonyl chloride, 1-Chloroethyl chloroformate, methyl chloroformate, and ethyl chloroformate.

Protecting groups can be added in a mole ratio to amine of between about 0.8 to about 1.2. For example, between about 0.95 and about 1.05. The chloroformate can be added in a slow controlled manner to reduce localized heating. Upon addition, the protected product can precipitate from solution. Suitable amines for protection comprise aromatic, primary, and secondary amines. The protection of the amines can be run neat or solvent such as tetrahydrofuran, 2 methyl tetrahydrofuran, acetonitrile, dichloromethane (DCM), pyridine, triethylamine, acetone, N,N-dimethylformamide (DMF), dimethyl sulfoxide (DMSO), tert-butyl methyl ether (TBME), cyclopentyl methyl ether (CPME) or some mixture in thereof. In embodiments, the solvents comprise 2-methyltetrahydrofuran (2Me-THF), tetrahydrofuran (THF), acetonitrile, and cyclopentyl methyl ether (CPME). In embodiments, solvents can have a water content of sub 50 ppm as determined by Karl Fischer titration.

The reaction can be run in the presence or absence of a base. Suitable bases comprise triethylamine, triethylamine diamine, dimethyl amino pyridine, 1,1,3,3-Tetramethylguanidine, Tetrabutylammonium hydroxide, or 1,8-bis(dimethylamino)naphthalene. In embodiments, bases comprise triethylamine diamine or Triethylamine. Bases can be added in a mol ratio to the amine of between about 0.2 and about 2. For example about 0.25 to about 1.1. Further, catalysts are often not required, however, common phase transfer catalysts can be used when solubility of the amine in the solvent of choice is an issue. Suitable catalysts include quaternary ammoniums such as tetrabutyl ammonium, benzyltriethylammonium, tetrabutyl ammonium, tetraethyl ammonium, cetyltrimethylammonium. Further, quaternary phosphoniums like Tetra-n-butylphosphonium are also suitable. Suitable counter anions comprise hydroxide, tosylate, fluoride, chloride or bromide. In embodiments, chloride and bromide.

Reaction vessels can glass or plastic round bottom flasks, or stainless steel or glass, jacketed or unjacketed vessels. Reaction temperatures can comprise about 0° C. to about 90° C. in one-degree increments. For example, between about 30° C. to about 90° C. in one-degree increments. For example, between about 50 and about 60° C. in one-degree increments. For example, reaction temperatures can comprise less than 0° C., about 0° C., about 5° C., about 10° C., about 15° C., about 20° C., about 25° C., about 30° C., about 35° C., about 40° C., about 45° C., about 50° C., about 55° C., about 60° C., about 70° C., about 75° C., about 80° C., about 90° C., about 100° C., or greater than about 100° C. The temperature can comprise a reaction temperature as by one of ordinary skill in the art depending on the reagents, solvent, and products.

The reaction is can be performed under an inert atmosphere such as argon or nitrogen. In some embodiments, the reaction is performed under nitrogen. Stirring can be performed with magnetic or mechanical stirring. Reaction times can vary; however, the range of reaction times is between about 1 and about 18 hours in about 30-minute increments, or until product has ceased precipitating from solution.

For example, reaction times can be determined by one of ordinary skill in the art by any method known in the art. For example, the reaction time can comprise less than 1 minute, about 1 min, about 5 min, about 10 min, about 15 min, about 20 min, about 25 min, about 30 min, about 45 min, about 1 hr, about 2 hrs, about 3 hrs, about 5 hrs, about 10 hrs, about 15 hrs, about 20 hrs, about 24 hrs, about 30 hrs, about 48 hrs, about 3 days, about 4 days, about 5 days, or greater than 5 days.

Purification procedures can comprise extractions, recrystallization, precipitation, chromatography techniques, dialysis, membrane techniques, absorption techniques or trituration. In embodiments, the purification procedures comprise membrane separation techniques, precipitation, trituration and recrystallization. For example, trituration by dissolving the product in a minimal amount of acetone or THF and slowly adding the minimal solution to 10× the organic volume of water and allowing the product to slowly crash out of solution over 12-18 hours. Pure products are collected by vacuum filtration. For water soluble products hexanes or MTBE is used as the anti or precipitation solvent.

For the enzymatic late-stage functionalization, enzymes that can bind, hydrolyze, or transesterify carbamate-containing molecules can be used. Suitable enzyme classes comprise proteases (EC 3.4), peptidases (EC 3.4), esterases (EC 3.1), amidases (EC 3.5), and ureases (3.5.1.5). For example, EC 3.4 and EC 3.5, alone or combinations in thereof. The quantity of enzymes employed in the reaction can comprise any range as determined by one of ordinary skill in the art by any method known in the art. For example, the employed quantity of enzymes can range from about 100 to about 1,000,000 international units (IU)/kg of substrate in increments of 1000, for example, the concentration can fall between about 100 to about 1000 IU/kg of activated substrate. For example, the activated substrate can be a carbamate protected amine. The concentration can be determined by one of ordinary skill in the art by any method known in the art.

Non-limiting, exemplary alcohol groups containing molecules for enzymatic transcarbamoylation can comprise monools such as methanol, ethanol, propanol (including isomers 1-propanol and 2-propanol), butanol (including isomers 1-butanol, 2-butanol, and tert-butanol), pentanol (including isomers 1-pentanol, 2-pentanol, and 3-pentanol), hexanol (including isomers 1-hexanol and 2-hexanol), heptanol (including isomers like 1-heptanol and 3-heptanol), octanol (with isomers like 1-octanol and 2-octanol), nonanol, decanol (including isomers like 1-decanol and 2-decanol), isoamyl alcohol, cyclohexanol, benzyl alcohol, allyl alcohol, propargyl alcohol, phenylethyl alcohol, methallyl alcohol, furfuryl alcohol, polyethylene glycol mono methyl ether, polypropylene glycol mono butyl ether, ethylene glycol mono-tert-butyl ether, and polyethylene glycol monoethyl ether. Diols such as ethylene glycol, 1,2-propanediol, 1,3-propanediol, 1,3-butanediol, 1,4-butanediol, poly(tetrahydrofuran) (poly THF), polyethylene glycol (PEG), polypropylene glycol, and polybutylene glycol. Polyols such as glycerol, sorbitol, mannitol, xylitol, glucose, fructose, and sucrose.

These enzymes can be introduced in solution form or as lyophilized solids. Non-nucleophilic and non-carbonyl containing buffers, such as phosphate, (3-(cyclohexylamino)-1-propanesulfonic acid) (CAPS), (2-(N-cyclohexylamino)ethanesulfonic acid) (CHES), boric acid, triethylamine, imidazole, (piperazine-N,N′-bis(2-ethanesulfonic acid)) (PIPES), or ammonium can be used. For example, phosphate, CAPS, triethylamine, or ammonium. Counter cations like lithium, sodium, and potassium can be used. In some embodiments, sodium and potassium are used. For counter anions, fluoride, chloride, bromide, or hydroxide can be used. For example, chloride or bromide can be utilized.

The concentration of buffer can comprise any concentration as determined by one of ordinary skill in the art by any method known in the art. For example, the range can be between about 10 and about 1000 mM in increments of about 10 mM. For example, the range can be from about less than 1 mM to greater than about 1000 mM. For example, the range can be between about 25 and about 200 mM. The suitable pH range for the reaction can be determined by one of ordinary skill in the art by any method known in the art. For example, the pH can be between about 8 and about 14 in increments of about 0.1. For example, the pH range can be between about 9 and about 12.

While not required, phase transfer catalysts when solubility in the alcohol/water mixture is limited, catalysts can comprise quaternary ammoniums such as tetrabutyl ammonium, benzyltriethylammonium, tetrabutyl ammonium, tetraethyl ammonium, cetyltrimethylammonium. Further, quaternary phosphoniums like Tetra-n-butylphosphonium can also suitable. Suitable counter anions can include, but are not limited to, hydroxide, tosylate, fluoride, chloride or bromide. For example, the anions comprise chloride and bromide. Reaction vessels comprise but are not limited to glass or plastic round bottom flasks, or stainless steel or glass, jacketed or unjacketed vessels.

Suitable temperatures of reaction comprise about 20° C. to about 120° C. in about one-degree increments. For example, between about 40 and about 80° C. in about one-degree increments. In some embodiments, the reaction is run under atmospheric conditions. In embodiments, the reaction can be performed under an inert atmosphere such as argon or nitrogen. In some embodiments, the reaction is performed under nitrogen.

Stirring can be performed with magnetic or mechanical stirring. Reaction times can vary; however, the range of reaction times is between about 1 and about 72 hours in about 30-minute increments, or until the product/starting material equilibrium appears to have been established according to HPLC or LCMS. Purification procedures can comprise extractions, recrystallization, precipitation, chromatography techniques, dialysis, membrane techniques, absorption techniques or trituration. In some embodiments, membrane separation techniques, precipitation, trituration and recrystallization can be used.

### REFERENCES CITED IN THIS EXAMPLE

(1) Castro, G. R. Properties of Soluble A-Chymotrypsin in Neat Glycerol and Water. Enzyme Microb. Technol. 2000.
(2) Stepankova, V.; Bidmanova, S.; Koudelakova, T.; Prokop, Z.; Chaloupkova, R.; Damborsky, J. Strategies for Stabilization of Enzymes in Organic Solvents. ACS Catal. 2013, 3 (12), 2823-2836. https://doi.org/10.1021/cs400684x.
(3) Del Amo, J.; Simón, D.; Ramos, M. J.; Rodriguez, J. F.; De Lucas, A.; Borreguero, A. M. Scaled-up and Economic Assessment Approach of the Split-Phase Glycolysis Process for the Recycling of Flexible Polyurethane Foam Wastes. J. Mater. Cycles Waste Manag. 2022, 24 (3), 1059-1071. https://doi.org/10.1007/s10163-022-01379-9.
(4) Deepa, P.; Jayakannan, M. Solvent-Free and Nonisocyanate Melt Transurethane Reaction for Aliphatic Polyurethanes and Mechanistic Aspects: Melt Transurethane Reaction. J. Polym. Sci. Part Polym. Chem. 2008, 46 (7), 2445-2458. https://doi.org/10.1002/pola.22578.
(5) Wolosz, D.; Parzuchowski, P. G.; Świderska, A. Synthesis and Characterization of the Non-Isocyanate Poly(Carbonate-Urethane)s Obtained via Polycondensation Route. Eur. Polym. J. 2021, 155, 110574. https://doi.org/10.1016/j.eurpolymj.2021.110574.
(6) Tanaka, K.; Kajiyama, N.; Ishikura, K.; Waga, S.; Kikuchi, A.; Ohtomo, K.; Takagi, T.; Mizuno, S. Determination of the Site of Disulfide Linkage between Heavy and Light Chains of Silk Fibroin Produced by Bombyx Mori. Biochim. Biophys. Acta.
(7) Zhou, C.-Z. Fine Organization of Bombyx Mori Fibroin Heavy Chain Gene. Nucleic Acids Res. 2000, 28 (12), 2413-2419. https://doi.org/10.1093/nar/28.12.2413.
(8) Nasr, K.; Meimoun, J.; Favrelle-Huret, A.; Winter, J. D.; Raquez, J.-M.; Zinck, P. Enzymatic Polycondensation of 1,6-Hexanediol and Diethyl Adipate: A Statistical Approach Predicting the Key-Parameters in Solution and in Bulk. Polymers 2020, 12 (9), 1907. https://doi.org/10.3390/polyml2091907.
(9) Hu, Y.; Daoud, W.; Cheuk, K.; Lin, C. Newly Developed Techniques on Polycondensation, Ring-Opening Polymerization and Polymer Modification: Focus on Poly(Lactic Acid). Materials 2016, 9 (3), 133. https://doi.org/10.3390/ma9030133.
(10) Ghosh, A. K.; Brindisi, M. Organic Carbamates in Drug Design and Medicinal Chemistry. J. Med. Chem. 2015, 58 (7), 2895-2940. https://doi.org/10.1021/jm501371s.
(11) Huang, L.-S.; He, F.; Huang, H.; Zhang, X.-Y.; Qi, S.-H. Carbamate Derivatives and Sesquiterpenoids from the South China Sea Gorgonian Melitodes Squamata. Beilstein J. Org. Chem. 2012, 8, 170-176. https://doi.org/10.3762/bjoc.8.18.

### Example 2

Examples of models where no enzymatic transcarbamoylation activity was observed:

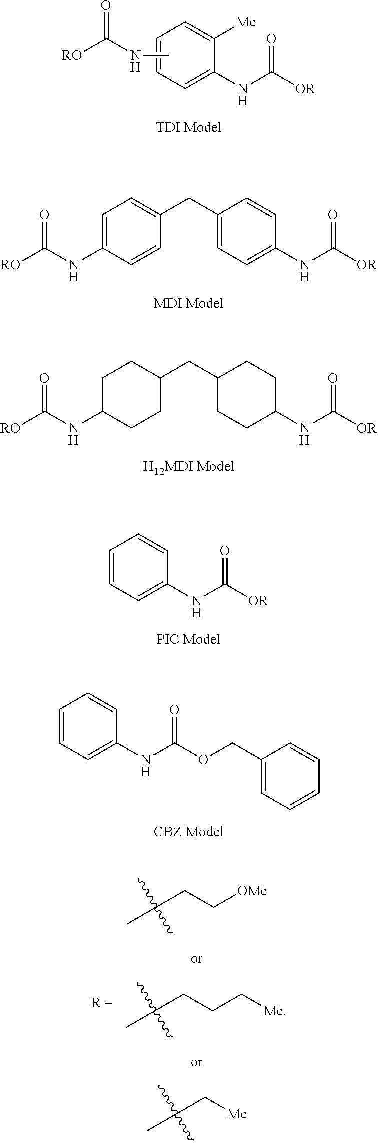

Non-limiting examples of models where enzymatic transcarbamoylation activity is observed:

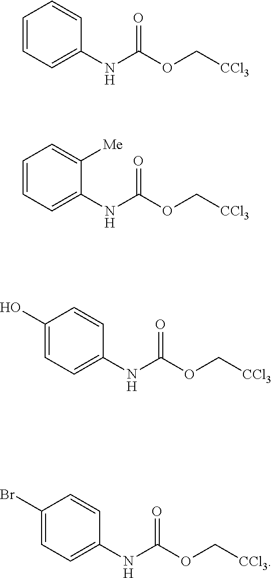

Non-Limiting examples alcohols where enzymatic transcarbamoylation activity is observed:

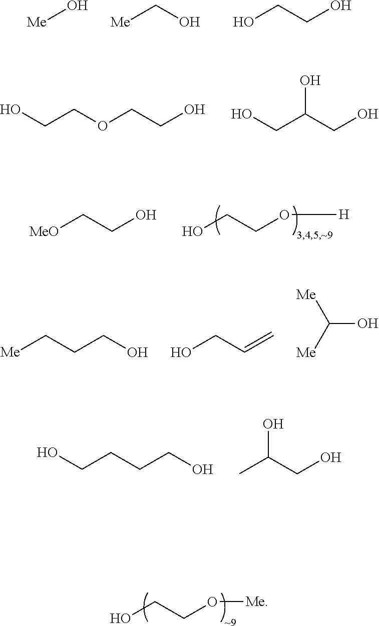

### Examples of Enzymes Screened (*Indicates Major Activity):

### Lipases:

Supplier=Millipore sigma—Lipase from Candidia sp. (Lipozyme CALB L); Lipase, immobilized on Immobead 150 from Pseudomonas cepacian; Lipase B Candida antarctica immobilized on Immobead 150, recombinant from Aspergillus oryzae; Lipase, immobilized on Immobead 150 from Rhizomucor miehei; Lipase from Candida rugosa; Lipase from wheat germ; Lipase from porcine pancreas; Amano Lipase PS, from Burkholderia cepacian; Lipase from Rhizopus niveus; Lipase from Rhizopus oryzae; and Lipase from Aspergillus niger.

Supplier=Novozymes—Lipozyme® TL IM (CAS No. 9001-62-1); Lipozyme® CALB L (CAS No. 9001-62-1); Lipozyme® TL 100 L (1,3 specific lipase originating from Thermomyces lanuginosus; CAS No.: 9001-62-1); NovoCor® AD L* (CAS No. 9001-62-1); Novozym® 435 (CAS No. 9001-62-1); Lipozyme® RM (CAS No. 9001-62-1); Novozym® 51032 (CAS No. 9001-62-1); Palatase® 20000 L (CAS No. 9001-62-1); Resinase® HT (CAS No. 9001-62-1); Novozymes Endoprotease Screening Kit (contains 6 endoprotease enzymes) (CAS No. 9014-01-1); and Novozymes Lipase Screening Kit (contains 9 lipase enzymes) (CAS No. 9001-62-1).

Proteases: Supplier=Millipore sigma—Bromelain from pineapple stem (CAS No. 37189-34-7); Supplier=Spectrum—Papain (CAS No. 9001-73-4); Supplier=TCI—Ficin (CAS No. 9001-33-6); Supplier=Spectrum—Alcalase® 2.4 L FG* (CAS No. 9014-01-1); Alcalase® 2.5 L* (CAS No. 9014-01-1); Esperase® 8.0 L** (CAS No. 9014-01-1); Neutrase® 0.8 L* (CAS No. 9080-56-2); Savinase® 12 T*(CAS No. 9014-01-1); and Savinase® 16 L** (CAS No. 9014-01-1).

Ureases: Supplier=Fischer—Urease (CAS No. 9002-13-5); Supplier=TCI—Urease from Jack Bean (CAS No. 9002-13-5)

Nitrilases: Supplier=Codexis—NIT-102 (CAS No. 9024-90-2); NIT-103 (CAS No. 9024-90-2); NIT-104 (CAS No. 9024-90-2); NIT-105 (CAS No. 9024-90-2); NIT-106 (CAS No. 9024-90-2); NIT-111 (CAS No. 9024-90-2); NIT-P1-118 (CAS No. 9024-90-2); NIT-P1-118 (CAS No. 9024-90-2); NIT-P1-118 (CAS No. 9024-90-2); NIT-P1-118 (CAS No. 9024-90-2); NIT-P1-118 (CAS No. 9024-90-2); and NIT-P1-118 (CAS No. 9024-90-2).

Laccase: Supplier=Millipore Sigma—Laccase from Aspergillus sp (CAS 80498-15-3).

Lit. Char. Urethanase: Plasmid supplied by Twist Bioscience

Ubrb from Ufarté et al., Discovery of carbamate degrading enzymes by functional metagenomics. PLoS One. 2017 Dec. 14; 12(12):e0189201. doi: 10.1371/journal.pone.0189201. PMID: 29240834.

### Example 3

### Additional References Cited Herein

(1) Darby, R. T.; Kaplan, A. M. Fungal Susceptibility of Polyurethanes. Appl. Microbiol. 1968, 16 (6), 900-905.
(2) Kim, J.; Choi, S. H.; Park, M. G.; Park, D. H.; Son, K.; Park, H. Biodegradation of Polyurethane by Japanese Carpenter Bee Gut-Associated Symbionts Xanthomonas Sp. HY-71, and Its Potential Application on Bioconversion. Environ. Technol. Innov. 2022, 102822.
(3) Gautam, R.; Bassi, A. S.; Yanful, E. K. Candida Rugosa Lipase-Catalyzed Polyurethane Degradation in Aqueous Medium. Biotechnol Lett 2007, 29, 1081-1086.
(4) Russell, J. R.; Huang, J.; Anand, P.; Kucera, K.; Sandoval, A. G.; Dantzler, K. W.; Hickman, D.; Jee, J.; Kimovec, F. M.; Koppstein, D.; Marks, D. H.; Mittermiller, P. A.; Nu, S. J.; Santiago, M.; Townes, M. A.; Vishnevetsky, M.; Williams, N. E.; Boulanger, L.; Bascom-slack, C.; Strobel, S. A. Biodegradation of Polyester Polyurethane by Endophytic Fungi. Appl. Environ. Microbiol. 2011, 77 (17), 6076-6084.
(5) Peng, Y.; Shih, Y.; Lai, Y.; Liu, Y.-Z.; Liu, Y.-T.; Lin, N.-C. Degradation of Polyurethane by Bacterium Isolated from Soil and Assessment of Polyurethanolytic Activity of a Pseudomonas Putida Strain. Env. Sci Pollut Res 2014, 21, 9529-9537.
(6) Biffinger, J. C.; Barlow, D. E.; Cockrell, A. L.; Cusick, K. D.; Hervey, W. J.; Fitzgerald, L. A.; Nadeau, L. J.; Hung, C. S.; Crookes-Goodson, W. J.; Russell Jr., J. N. The Applicability of Impranil® DLN for Gauging the Biodegradation of Polyurethanes. Polym. Degrad. Stab. 2015, 120, 178-185.
(7) Schmidt, J.; Wei, R.; Oeser, T.; Andre, L.; Breite, D.; Schulze, A.; Zimmermann, W. Degradation of Polyester Polyurethane by Bacterial Polyester Hydrolases. Polymers (Basel). 2017, 9 (65), 1-10.
(8) Magnin, A.; Pollet, E.; Perrin, R.; Ullmann, C.; Persillon, C.; Phalip, V.; Avérous, L. Enzymatic Recycling of Thermoplastic Polyurethanes: Synergistic Effect of an Esterase and an Amidase and Recovery of Building Blocks. Waste Manag. 2019, 85, 141-150.
(9) Zhang, K.; Hu, J.; Yang, S.; Xu, W.; Wang, Z.; Zhuang, P.; Grossart, H.; Luo, Z. Biodegradation of Polyester Polyurethane by the Marine Fungus Cladosporium Halotolerans 6UPA1. J. Hazard. Mater. 2022, 437 (April), 129406.
(10) Ruiz, C.; Main, T.; Hilliard, N. P.; Howard, G. T. Purification and Characterization of Two Polyurethanase Enzymes from Pseudomonas Chlororaphis. Int. Biodeterior. Biodegrad. 32 1999, 43-47.
(11) Nakajima-kambe, T.; Shigeno-Akutsu, Y.; Nomura, N.; Onuma, F.; Nakahara, T. Microbial Degradation of Polyurethane, Polyester Polyurethanes and Polyether Polyurethanes. Appl Microbiol Biotechnol 1999, 51, 134-140.
(12) Shah, A. A.; Hasan, F.; Akhter, J. I.; Hameed, A.; Ahmed, S. Degradation of Polyurethane by Novel Bacterial Consortium Isolated from Soil. Ann. Microbiol. 2008, 58 (3), 381-386.
(13) Matsumiya, Y.; Murata, N.; Tanabe, E.; Kubota, K.; Kubo, M. Isolation and Characterization of an Ether-Type Polyurethane-Degrading Micro-Organism and Analysis of Degradation Mechanism by Alternaria Sp. J. Appl. Microbiol. 2010, 108, 1946-1953.
(14) Shah, Z.; Krumholz, L.; Aktas, D. F.; Hasan, F.; Khattak, M.; Shah, A. A. Degradation of Polyester Polyurethane by a Newly Isolated Soil Bacterium, Bacillus Subtilis Strain MZA-75. Biodegradation 2013, 24, 865-877. protect-us.mimecast.com/s/6NsaC68m51cBvr3yCj3byz?domain=doi.org.
(15) Lando, G. A.; Marconatto, L.; Kessler, F.; Lopes, W.; Schrank, A.; Vainstein, M. H.; Weilbel, D. E. UV-Surface Treatment of Fungal Resistant Polyether Polyurethane Film-Induced Growth of Entomopathogenic Fungi. Int. J. Mol. Sci. 2017, 18, 1536.
(16) Oprea, S.; Potolinca, V. O.; Gradinariu, P.; Oprea, V. Biodegradation of Pyridine-Based Polyether Polyurethanes by the Alternaria Tenuissima Fungus. J. Appl. Polym. Sci. 2018, 2, 1-10.
(17) Gaytin, I.; Sinchez-reyes, A.; Burelo, M.; Vargas-suárez, M.; Liachko, I.; Press, M.; Sullivan, S.; Cruz-Gómez, J. M.; Loza-tavera, H. Degradation of Recalcitrant Polyurethane and Xenobiotic Additives by a Selected Landfill Microbial Community and Its Biodegradative Potential Revealed by Proximity Ligation-Based Metagenomic Analysis. Front. Microbiol. 2020, 10, 2986.
(18) Pathirana, R. A.; Seal, K. J. Studies on Polyurethane Deteriorating Fungi Part 3. Physico-Mechanical and Weight Changes during Fungal Deterioration. Int. Biodeterior. 1985, 21 (1), 41-50.
(19) Jansen, B.; Schumacher-Perdreau, F.; Peters, G.; Pulverer, G. Evidence for Degradation of Synthetic Polyurethanes by Staphylococcus Epidermidis. Zentralbl Bakteriol 1991, 45, 36-45.
(20) Crabbe, J. R.; Walz, S. E.; Montgomery, M. T. PSEUDOMONAS CHLORORAPHIS MICROORGANISM, POLYURETHANE DEGRADING ENZYME OBTAINED THEREFROM AND METHOD OF USING ENZYME, 1995.
(21) Nomura, N.; Shigeno-Akutsu, Y.; Nakajima-kambe, T.; Nakahara, T. Cloning and Sequence Analysis of a Polyurethane Comamonas Acidovorans TB-35 Esterase. J. Ferment. Bioeng. 1998, 86 (4), 339-345.
(22) Akutsu, Y.; Nakajima-kambe, T.; Nomura, N. Purification and Properties of a Polyester Polyurethane-Degrading Enzyme from Comamonas Acidovorans TB-35. Appl. Environ. Microbiol. 1998, 64 (1), 62-67.
(23) Nakajima-kambe, T.; Onuma, F.; Akutsu, Y.; Nakahara, T. Determination of the Polyester Polyurethane Breakdown Products and Distribution of the Polyurethane Degrading Enzyme of Comamonas Acidovorans Strain TB-35. J. Ferment. Bioeng. 1997, 83 (5), 456-460.
(24) Kim, Y. D.; Kim, S. C. Effect of Chemical Structure on the Biodegradation of Polyurethanes under Composting Conditions. Polym. Degrad. Stab. 1998, 62, 343-352.
(25) Smith, R.; Oliver, C.; Williams, D. F. The Enzymatic Degradation of Polymers in Vitro. J. Biomed. Mater. Res. 1987, 21, 991-1003.
(26) Wales, D. S.; Sagar, B. F. Mechanistic Aspects of Polyurethane Biodeterioration. In Biodeterioration 7; Houghton, D. R., Smith, R. N., Eggins, H. O. W., Eds.; Springer Netherlands: Dordrecht, 1988; pp 351-358.
(27) Santerre, J. P.; Labow, R. S.; Duguay, D. G.; Erifle, D.; Adams, G. A. Biodegradation Evaluation of Polyether and Polyester-Urethanes with Oxidative and Hydrolytic Enzymes. J. Biomed. Mater. Res. 1994, 28, 1187-1199.
(28) Schubert, M. A.; Wiggins, M. J.; Schaefeq, M. P.; Hiltner, A.; Anderson, J. M. Oxidative Biodegradation Mechanisms of Biaxially Strained Poly (Etherurethane Urea) Elastomers. J. Biomed. Mater. Res. 1995, 29, 337-347.
(29) Stokes, K.; Mcvenes, R.; Anderson, J. M. Polyurethane Elastomer Biostability. J. Biomater. Appl. 1995, 9, 321-354. protect-us.mimecast.com/s/yHFnCjRvlrsvXn6RszYaru?domain=doi.org.
(30) Labow, R. S.; Erfle, D. J.; Santerre, J. P. Elastase-Induced Hydrolysis of Synthetic Solid Substrate: Poly(Ester-Urea-Urethane) and Poly(Ether-Urea-Urethane). Biomaterials 1996, 17 (24), 2381-2388.
(31) McCarthy, S. J.; Meijs, G. F.; Mitchell, N.; Gunatillake, P. A.; Heath, G.; Brandwood, A.; Schindhelm, K. In-Vivo Degradation of Polyurethanes: Transmission-FTIR Microscopic Characterization of Polyurethanes Sectioned by Cryomicrotomy. Biomaterials 1997, 18 (21), 1387-1409.
(32) Christenson, E. M.; Patel, S.; Anderson, J. M.; Hiltner, A. Enzymatic Degradation of Poly (Ether Urethane) and Poly (Carbonate Urethane) by Cholesterol Esterase. Biomaterials 2006, 27, 3920-3926.
(33) Mcbane, J. E.; Santerre, P. J.; Labow, R. S. The Interaction between Hydrolytic and Oxidative Pathways in Macrophage-Mediated Polyurethane Degradation. J. Biomed. Mater. Res. 2007, 984-994.
(34) Christenson, E. M.; Anderson, J. M.; Hiltner, A. Biodegradation Mechanisms of Polyurethane Elastomers. Corros. Eng. Sci. Technol. 2007, 42 (4), 312-323. protect-us.mimecast.com/s/RnfSCmZ2oxFr3j0WUkZc1R?domain=doi.org.
(35) Hong, Y.; Guan, J.; Fujimoto, K. L.; Hashizume, R.; Pelinescu, A. L.; Wagner, W. R. Biomaterials Tailoring the Degradation Kinetics of Poly (Ester Carbonate Urethane) Urea Thermoplastic Elastomers for Tissue Engineering Scaffolds. Biomaterials 2010, 31 (15), 4249-4258. protect-us.mimecast.com/s/xFzmCn5YpyHY27ymTXIQQh?domain=doi.org.
(36) Ratner, B. D.; Gladhill, K. W.; Horbett, T. A. Analysis of in Vitro Enzymatic and Oxidative Degradation of Polyurethanes. J. Biomed. Mater. Res. 1988, 22, 509-527.
(37) Sharma, S. M.; Szycher, M. M. Blood Compatible Materials and Devices: Perspectives towards the 21st Century.; Technomic Publishing Co., 1991.
(38) Siciliano, A. A.; Szycher, M. An Assessment of 2,4 TDA Formation from Surgitek Polyurethane Foam under Simulated Physiological Conditions. J. Biomater. Appl. 1991, 5, 323-336.
(39) Bouvier, M.; Chawla, A. S.; Hinberg, I. In Vitro Degradation of a Poly (Ether Urethane) by Trypsin. J. Biomed. Mater. Res. 1991, 25, 773-789.
(40) Wu, Y.; Zhao, Q.; Anderson, J. M.; Hiltner, A.; Lodoen, G.; Payet, C. R. Effect of Some Additives on the Biostability of a Poly(Etherurethane) Elastomer. J. Biomed. Mater. Res. 1991, 25, 725-739.
(41) Tyler, B. J.; Ratner, B. D. Variations between Biomer Lots. 2: The Effect of Differences between Lots on in Vitro Enzymatic and Oxidative Degradation of a Commercial Polyurethane. J. Biomed. Mater. Res. 1993, 27, 327-334.
(42) Hergenrother, R. W.; Wabers, H. D.; Cooper, S. L. Effect of Hard Segment Chemistry and Strain on the Stability of Polyurethanes: In Vivo Biostability. Biomaterials 1993, 14 (6), 449-458.
(43) Santerre, J. P.; Labow, R. S.; Adams, G. A. Enzyme-Biomaterial Interactions: Effect of Biosystems on Degradation of Polyurethanes. J. Biomed. Mater. Res. 1993, 27, 97-109.
(44) Cregut, M.; Bedas, M.; Durand, M.-J.; Thouand, G. New Insights into Polyurethane Biodegradation and Realistic Prospects for the Development of a Sustainable Waste Recycling Process. Biotechnol. Adv. 2013, 31 (8), 1634-1647. protect-us.mimecast.com/s/U2d0Co2vqzTORrovS9s2s-?domain=doi.org.
(45) Mahajan, N.; Gupta, P. New Insights into the Microbial Degradation of Polyurethanes. RSC Adv. 2015, 5, 41839-41854.
(46) Magnin, A.; Pollet, E.; Phalip, V.; Avérous, L. Evaluation of Biological Degradation of Polyurethanes. Biotechnol. Adv. 2020, 39, 107457.
(47) Jin, X.; Dong, J.; Guo, X.; Ding, M. Current Advances in Polyurethane Biodegradation. Soc. Ind. Chem. 2021, No. December, 1-9.
(48) Liu, J.; He, J.; Xue, R.; Xu, B.; Qian, X.; Xin, F.; Blank, L. M.; Zhou, J.; Wei, R.; Dong, W.; Jiang, M. Biodegradation and Up-Cycling of Polyurethanes: Progress, Challenges, and Prospects. Biotechnol. Adv. 2021, 48 (December 2020), 107730.
(49) Sari, P. S.; Baneesh, N. S.; Reghunadhan, A.; Abraham, J.; Thomas, S. Recycling of Polyurethanes. Polyurethane Chem. Renew. Polyols Isocyanates, Part 15—Recycl. Polyurethanes 2021.
(50) Zambelli, B.; Musiani, F.; Benini, S.; Ciurli, S. Chemistry of Ni2+ in Urease: Sensing, Trafficking, and Catalysis. Acc. Chem. Res. 2011, 44 (7), 520-530.
(51) Fich, E. A.; Segerson, N. A.; Rose, J. K. C. The Plant Polyester Cutin: Biosynthesis, Structure, and Biological Roles. Annu. Rev. Plant Biol. 2016, 67, 207-233.
(52) Lu, H.; Diaz, D. J.; Czarnecki, N. J.; Zhu, C.; Kim, W.; Shroff, R.; Acosta, D. J.; Alexander, B. R.; Cole, H. O.; Zhang, Y.; Lynd, N. A.; Ellington, A. D.; Alper, H. S. Machine Learning-Aided Engineering of Hydrolases for PET Depolymerization. Nature 2022, 604, 662-667.
(53) Knott, B. C.; Erickson, E.; Allen, M. D.; Gado, J. E.; Graham, R.; Kearns, F. L.; Rorrer, N. A.; Szostkiewicz, C. J.; Copié, V.; Payne, C. M.; Woodcock, H. L. Characterization and Engineering of a Two-Enzyme System for Plastics Depolymerization. PNAS 2020, 117 (41), 25476-25485.
(54) Wilkes, G. L.; Dziemianowicz, T. S.; Ophir, Z. H. Thermally Induced Time Dependence of Mechanical Properties in Biomedical Grade Polyurethanes. J. Biomed. Mater. Res. 1979, 13, 189-206.
(55) Keer, L. De; Steenberge, P. Van; Reyniers, M.; Gryn, G.; Aitken, H. M.; Coote, M. L. New Mechanism for Autoxidation of Polyolefins: Kinetic Monte Carlo Modelling of the Role of Short-Chain Branches, Molecular Oxygen and Unsaturated Moieties. Polym. Chem. 2022, 13, 3304-3314.
(56) Takahara, A.; Coury, A. J.; Hergenrother, R. W.; Cooper, S. L. Effect of Soft Segment Chemistry on the Biostability of Segmented Polyurethanes. I. In Vitro Oxidation. J. Biomed. Mater. Res. 1991, 25, 341-356.
(57) Takahara, A.; Hergenrother, R. W.; Coury, A. J.; Coopert, S. L. Effect of Soft Segment Chemistry on the Biostability of Segmented Polyurethanes. II. In Vitro Hydrolytic Degradation and Lipid Sorption. J. Biomed. Mater. Res. 1992, 26, 801-818.
(58) Nakajima-kambe, T. Isolation of a Bacterium That Degrades Urethane Compounds and Characterization of Its Urethane Hydrolase. Appl Microbiol Biotechnol 2006, 70, 422-429.
(59) Nakajima-kambe, T.; Shigeno-Akutsu, Y. URETHANE-BOND-DEGRADING BACTERIA. U.S. Ser. No. 00/754,7537B2, 2009.
(60) Magnin, A.; Pollet, E.; Avérous, L. Characterization of the Enzymatic Degradation of Polyurethanes. In Methods in Enzymology; 2021; Vol. 648, pp 317-336.
(61) Li, X.; Liu, W.; Li, Y.; Lan, W.; Zhao, D.; Wu, H.; Feng, Y.; He, X.; Li, Z.; Li, J.; Luo, F.; Tan, H. Mechanically Robust Enzymatic Degradable Shape Memory Polyurethane Urea with Rapid Recovery Response Induced by NIR. J. Mater. Chem. B 2020, 8 (23), 5117-5130.
(62) Calleros, E. L.; Simonovsky, F. I.; Garty, S.; Ratner, B. D. Crosslinked, Biodegradable Polyurethanes for Precision-Porous Biomaterials: Synthesis and Properties. J. Appl. Polym. Sci. 2020, 48943, 1-16.
(63) Gunawan, N. R.; Tessman, M.; Schreiman, A. C.; Simkovsky, R.; Samoylov, A. A.; Neelakantan, N. K.; Bemis, T. A.; Burkart, M. D.; Pomeroy, R. S.; Mayfield, S. P. Rapid Biodegradation of Renewable Polyurethane Foams with Identification of Associated Microorganisms and Decomposition Products. Bioresour. Technol. Reports 2020, 11, 100513.
(64) US007547537B2
(65) Branson et al., Urethanases for the Enzymatic Hydrolysis of Low Molecular Weight Carbamates and the Recycling of Polyurethanes, Volume 62, Issue 9, Feb. 20, 2023, e202216220

### Example 4

### Promiscuous Biocatalytic Transcarbamoylation: An Enzymatic Route to a More Circular Polyurethane Economy

In a development toward sustainable chemistry, we describe a method of using wild-type enzymes for the promiscuous catalysis of late-stage functionalization of compounds. The late-stage functionalized described herein can be used, as a non-limiting example, in the development of pharmaceutical and agricultural compounds. The innovations described herein mark a significant departure from conventional transcarbamoylation methods that typically employ strong Lewis acids and highly alkaline conditions, which limits the reaction's applicability to alkaline-sensitive functional groups.

Leveraging the unique capabilities of these enzymes, we have achieved high conversion yields. For example, about 99% conversion and about 91% yield of transcarbamoylation product in less than 12 hours under conditions with compounds described herein. Further, we can achieve high substrate loads. For example, we can achieve substrate loading of about 100 mM. The process is not limited to a single alcohol or glycol and has been demonstrated on at least 16 different alcohols. The newly discovered enzymatic showcases a route for the late-stage functionalization of compounds in various sectors including, but not limited to, cosmetics, pharmaceuticals, and agrochemicals.
The enzyme mining process can comprise, for example, identifying targets (e.g., protein data bank, uniport, foldseek), characterizing in silico (e.g., Alphafold, DiffDock, AutoDock), and screening enzymes (e.g., Twist Bioscience, 96-well screens, LC-MS).
Transcarbamoylation activity was not observed among most of the enzymes screened, but we identified some as described herein.
We were able to obtain high yields of transcarbamoylation products as determined by LCMS.

### EQUIVALENTS

Those skilled in the art will recognize, or be able to ascertain, using no more than routine experimentation, numerous equivalents to the specific substances and procedures described herein. Such equivalents are considered to be within the scope of this invention and are covered by the following claims.

What is claimed is:

1. A method of late-stage functionalization, the method comprising:
selecting a compound with a nucleophilic moiety;
reacting the nucleophilic moiety with a protecting group reagent, thereby producing an activated compound;
subjecting the activated compound to at least one functionalization molecule and at least one hydrolase, wherein the functionalization molecule contains a nucleophilic group, thereby producing a transcarbamoylation reaction product.

2. The method of claim 1, wherein the method of late-stage functionalization comprises a reaction according to: 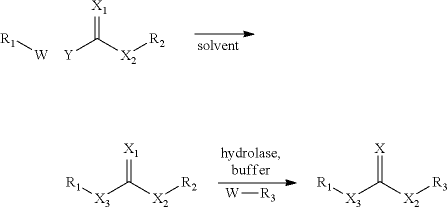
wherein R₁ is an organic moiety;
R₂ is selected from the group consisting of -Ph, -Me, —CCH₂CCHCCH₂, -Et, —Pr, -Bu, or —CH₂Ph;
R₃ is an organic moiety;
W is selected from the group consisting of —NH₂, —SH, —OH or —NHR₄, wherein R₄ is selected from the group consisting of alkyl, alkenyl, alkynyl, aryl, heteroaryl, cycloalkyl, heterocycloalkyl, haloalkyl, hydroxyalkyl, alkoxyalkyl, carboxyalkyl, aminoalkyl, or a substituted derivative thereof;
X₁ is selected from the group consisting of —O—, —S—, or —NH—;
X₂ is selected from the group consisting of —O—, —S—, or —NH;
X₃ is selected from the group consisting of —O—, —S—, —NH—, or —NR₄—, wherein R₄ is selected from the group consisting of alkyl, alkenyl, alkynyl, aryl, heteroaryl, cycloalkyl, heterocycloalkyl, haloalkyl, hydroxyalkyl, alkoxyalkyl, carboxyalkyl, aminoalkyl, or a substituted derivative thereof;
wherein X₃ is —O— when W is —OH,
wherein X₃ is —S— when W is —SH, and
wherein X₃ is —NH when W is NH₂; and
Y is selected from the group consisting of —Cl, —OPh, —OMe, —OCCH₂CCHCCH₂, —OEt, —OPr, —OBu, or —OCH₂Ph.

3. The method of claim 2, wherein the R₁ is selected from the group consisting of alkyl, alkenyl, alkynyl, aryl, heteroaryl, cycloalkyl, heterocycloalkyl, haloalkyl, hydroxyalkyl, alkoxyalkyl, carboxyalkyl, aminoalkyl, or a substituted derivative thereof.

4. The method of claim 2, wherein R₃ is selected from the group consisting of alkyl, alkenyl, alkynyl, aryl, heteroaryl, cycloalkyl, heterocycloalkyl, haloalkyl, hydroxyalkyl, alkoxyalkyl, carboxyalkyl, aminoalkyl, or a substituted derivative thereof.

5. The method of claim 1, wherein the hydrolase is selected from the group consisting of NovoCor® AD L, Alcalase® 2.4 L FG, Alcalase® 2.5 L, Esperase® 8.0 L, Savinase® 16 L, or a combination thereof.

6. The method of claim 1, wherein the nucleophilic moiety is selected from the group consisting of an amine, an alcohol, or a thiol.

7. The method of claim 1, wherein the protecting group reagent is selected from the group consisting of t-Butyloxycarbonyl chloride, di-tert-butyl decarbonate, benzyloxycarbonyl chloride, fluorenylmethyloxycarbonyl chloride, allyloxycarbonyl chloride, 2,2,2-Trichloroethoxycarbonyl chloride (Troc-Cl), methyl chloroformate, ethyl chloroformate, 1-Chloroethyl chloroformate, trichloromethyl chloroformate, chloromethyl chloroformate, dimethyl carbonate, (1S)-(+)-Menthyl chloroformate, phenyl chloroformate, or a combination thereof.

8. The method of claim 1, wherein the functionalization molecule is selected from the group consisting of an alcohol, a polyol, or a combination thereof.

9. The method of claim 8, wherein the alcohol is selected from the group consisting of methanol, ethanol, propanol, butanol, pentanol, hexanol, heptanol, octanol, nonanol, decanol, isoamyl alcohol, cyclohexanol, benzyl alcohol, allyl alcohol, propargyl alcohol, phenylethyl alcohol, methallyl alcohol, furfuryl alcohol, an isomer thereof, or a combination thereof.

10. The method of claim 8, wherein the polyol is selected from the group consisting of a diol, a triol, a sugar, a sugar alcohol, a polymeric polyol, or any combination thereof.

11. The method of claim 10, wherein the diol is selected from the group consisting of ethylene glycol, 1,2-propanediol, 1,3-propanediol, 1,3-butanediol, 1,4-butanediol.

12. The method of claim 10, wherein the triol is glycerol.

13. The method of claim 10, wherein the sugar alcohol is selected from the group consisting of sorbitol, mannitol, xylitol, or any combination thereof.

14. The method of claim 10, wherein the sugar is selected from the group consisting of glucose, fructose, maltose, or any combination thereof.

15. The method of claim 10, wherein the polymeric polyol is selected from the group consisting of polyethylene glycol, polyethylene glycol mono methyl ether, polypropylene glycol mono ethyl ether, polypropylene glycol, polypropylene glycol mono butyl ether, polypropylene glycol mono methyl ether, ethylene glycol mono-tert-butyl ether, polyethylene glycol monoethyl ether, or any combination thereof.

16. The method of claim 1, wherein the late-stage functionalized molecule is selected from the group consisting of a carbamate, a carbonate, a thiocarbonate, a urea, or a thiocarbamate.

17. The method of claim 1, further comprising purifying the late-stage functionalized molecule.

18. The method of claim 1, wherein the method does not require a base catalyst.

19. A compound produced by the method of claim 1.

20. A pharmaceutical composition comprising the compound of claim 19 and a pharmaceutically acceptable excipient.
